(19) United States
(12) Patent Application Publication
(10) Pub. No.: US 2014/0255353 A1
(43) Pub. Date: Sep. 11, 2014

(54) COMPOSITIONS, KITS AND METHODS FOR TREATING OBESITY, DIABETES AND HYPERGLYCEMIA

(71) Applicants: Keith Webster, Key Biscayne, FL (US); Nikhil Adi, Omaha, NE (US)

(72) Inventors: Keith Webster, Key Biscayne, FL (US); Nikhil Adi, Omaha, NE (US)

(73) Assignee: UNIVERSITY OF MIAMI, Miami, FL (US)

(21) Appl. No.: 14/241,657

(22) PCT Filed: Aug. 29, 2012
(86) PCT No.: PCT/US2012/052799

§ 371 Date: May 27, 2014

Related U.S. Application Data
(60) Provisional application No. 61/528,453, filed on Aug. 29, 2011.

(51) Int. Cl. C12N 15/86

(52) U.S. Cl. CPC C12N 15/86
USPC 424/93.6; 514/44 R

(57) ABSTRACT

Described herein are compositions, kits and methods for treating and preventing obesity and overweight, as well as treating and preventing hyperglycemia, in a subject (e.g., a human), based on the discoveries that reduced expression of miR-205 may be a cause of obesity, and that miR-411 regulates gluconeogenic genes by targeting transcription factors, respectively. The compositions, kits and methods for treating and preventing obesity and overweight involve increasing expression of one or more microRNAs (e.g., miR-205) involved in adipogenesis, and provide a new therapy for treating patients that have eating disorders and/or are predisposed to obesity, and that are obese or overweight. The compositions, kits and methods described herein for treating and preventing hyperglycemia involve increasing expression of miR-411 and provide a new therapy for treating hyperglycemia associated with, for example, type 2 diabetes.

### CROSS-REFERENCE TO RELATED APPLICATIONS

This application claims the benefit of Provisional Application Ser. No. 61/528,453 filed Aug. 29, 2011, which is herein incorporated by reference in its entirety.

### FIELD OF THE INVENTION

The invention relates generally to the fields of medicine and gene therapy. More particularly, the invention relates to compositions, kits and methods for treating and preventing obesity, overweight, hyperglycemia, and other disorders associated with type 2 diabetes.

### BACKGROUND

The epidemic of obesity and type 2 diabetes poses a challenge to our health care system and an enormous burden of morbidity and early mortality that is predicted to affect up to 20% of Americans by 2030. In analyses carried out for World Health Report 2002, approximately 60% of diabetes and 20% of ischemic heart disease globally are attributable to obesity. Central obesity involves deposition of fat around the trunk and in visceral adipose tissue. Both obesity and abdominal obesity increased in men, and abdominal obesity increased in women between 1999 and 2008 and carries a higher risk of cardiovascular disease, dyslipidemia and type 2 diabetes. In obese individuals, the increase in adiposity results from increases in the number and size of adipocytes, while the degree of hypertrophy relative to hyperplasia influences the level of body fat and the metabolic consequences of obesity.

Hyperglycemia is another metabolic disorder that poses a challenge to the health care system. The liver is the primary organ for glucose and lipid metabolism. During fasting, blood glucose is maintained by the breakdown of hepatic glycogen followed by gluconeogenesis. In the postprandial state, insulin action in the liver turns off both glycogenolysis and gluconeogenesis and activates glycogenesis and lipogenesis to store the excess glucose. Tight insulin-regulation of hepatic glucose production is essential to maintain normoglycemia and dysregulation caused by insulin resistance is the most prevalent metabolic abnormality in the developed world.

Accordingly, improved treatment and prevention strategies for these disorders are needed. Novel methods and therapeutics to suppress gluconeogenesis could provide new treatment strategies to regulate and reduce hyperglycemia in subjects that cannot be controlled by current pharmacology and/or lifestyle, while new methods and therapeutics for the suppression of adipose deposition and expansion could provide new treatment strategies for highly susceptible patients especially those that do not respond to current drugs and lifestyle modification.

### SUMMARY

Described herein are compositions, kits and methods for treating and preventing obesity, overweight and hyperglycemia in a subject (e.g., a human patient). The compositions, kits and methods described herein for treating and preventing obesity and overweight involving increasing levels of one or more microRNAs (also referred to herein as miRNAs and miRs) involved in adipogenesis (e g , miR-205) provide a new therapy for treating patients, including adults and children, that have eating disorders and/or are predisposed to obesity, and that are obese or overweight. These compositions, kits and methods may be used to treat all overweight or obese patients, particularly patients suffering from obesity associated with type 2 diabetes. The discovery that miR-205 targets adipogenesis and is down-regulated in visceral adipose of obese mice is entirely novel. The mouse model that was used is also novel and the only mouse model available that closely resembles the human obese/type 2 diabetic phenotype in multiple respects including polygenic nature of the predisposition, milder obese phenotype and strict diet and age-dependent phenotype. The compositions, kits and methods described herein for treating and preventing hyperglycemia involving increasing miR-41 1 levels provide a new therapy for treating hyperglycemia associated with type 2 diabetes. These compositions, kits and methods may be used to treat hyperglycemia in all patients, particularly hyperglycemia associated with obesity and type 2 diabetes. The discovery that miR-411 targets gluconeogenic genes and is down-regulated in the liver of diabetic mice is entirely novel, as is use of the aforementioned mouse model for examining hyperglycemia.

Accordingly, described herein is a method of treating or preventing obesity or overweight in a subject (e.g. a human). The method includes administering to the subject a composition including at least one agent that increases expression of at least one miR such as, for example, miR-200a, miR-200b, miR-200c, miR-143, and miR-205, in a therapeutically effective amount for treating or preventing obesity or overweight in the subject, and a pharmaceutically acceptable carrier. In the method, administration of the composition reduces adiposity levels and adipose production in the subject. In one embodiment, the at least one agent increases expression of miR-205 and is pre-miR-205. In this embodiment, pre-miR-205 can be an oligonucleotide, or present in a viral vector (e.g., a viral vector within a recombinant virion) or a nanoparticle. In another embodiment, the at least one agent that increases expression of at least one miR is a vector expressing the at least one miR (e.g., a viral vector expressing miR-205). In the method, the composition can further include a tissue-specific or cell-specific targeting molecule. The subject is typically a human, and may be overweight or obese. The subject may have a genetic predisposition to obesity and/or type 2 diabetes.

Also described herein is a composition including at least one agent that increases expression of at least one miR such as, for example, miR-200a, miR-200b, miR-200c, miR-143, and miR-205 in a subject in a therapeutically effective amount for treating or preventing obesity or overweight in a subject, and a pharmaceutically acceptable carrier. The at least one agent typically includes a viral vector expressing a pre-miR to the at least one miR or a viral vector expressing the at least one miR. The viral vector may be present in a recombinant virion. In an embodiment of increasing expression of miR-205, for example, the composition may further include a second agent that increases expression of miR-205, wherein the second agent is a PPAR gamma antagonist.

Yet further described herein is a method of treating or preventing hyperglycemia in a subject (e.g., a human) including administering to the subject a composition including an agent that increases expression of miR-411 in the subject in a therapeutically effective amount for treating or preventing hyperglycemia. In the method, administration of the composition reduces tissue glucose production and increases glucose removal in the subject. Administration of the composition may also reduce hepatic HNF4a expression in the subject. In some embodiments, the composition is administered to the subject's liver. The subject may be obese, overweight, have diabetes, be pre-diabetic, have acute hyperglycemia, chronic hyperglycemia, stress hyperglycemia, etc. In one embodiment, the at least one agent that increases expression of miR-411 is a vector (e.g., a viral vector) expressing miR-411. In another embodiment, the agent that increases expression of miR-411 is pre-miR-411. In this embodiment, pre-miR-411 can be an oligonucleotide, or present in a viral vector (e.g., a viral vector within a recombinant virion) or a nanoparticle. A composition may further include a tissue-specific or cell-specific targeting molecule.

Still further described herein is a composition including at least one agent that increases expression of miR-411 in a subject in a therapeutically effective amount for treating or preventing hyperglycemia in a subject, and a pharmaceutically acceptable carrier. The at least one agent includes a viral vector expressing pre-miR-411 or a viral vector expressing miR-411.

Unless otherwise defined, all technical terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid), and chemically-modified nucleotides. A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The terms include, e.g., a recombinant nucleic acid molecule incorporated into a vector, a plasmid, a virus, or a genome of a prokaryote or eukaryote. Examples of purified nucleic acids include cDNAs, micro-RNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

By the term “gene” is meant a nucleic acid molecule that codes for a particular protein, or in certain cases, a functional or structural RNA molecule.

As used herein, the phrase “transduced with at least one nucleic acid” means any method of transferring a nucleic acid into a cell; such methods include but are not necessarily limited to transfer of naked DNA in the form of oligonucleotides with or without chemical modifications and with or without optimized delivery systems for oligonucleotides including calcium phosphate, lipids (e.g., liposomes, lipifectin reagents), nanoparticles, etc. Transferring a nucleic acid into a cell can occur after cloning of a nucleic acid into plasmid or viral vectors, the latter to include, for example, Adeno-associated Virus (AAV), adenovirus and all categories of retrovirus (e.g., lentivirus, Human Immunodeficiency Virus (HIV) and related viruses). “Transduction” can also be used to refer to cells that have been infected with a virus (virions, particles) that contains a vector including a nucleic acid sequence to be transferred into the cell.

By the terms “microRNA,” “miRNA” and “miR” are meant short (average 22 nucleotides) non-coding RNAs miRs are post-transcriptional regulators that bind to complementary sequences on target messenger RNA transcripts and usually repress translation or cause mRNA target degradation with gene silencing. miRs may be endogenous or synthetic.

As used herein, the term “pre-miR” means precursor miRs. pre-miRs include single stranded, double stranded, partially double stranded and hairpin structured chemically modified nucleic acids that augment the levels of a target miR in a sequence-dependent manner Usually, pre-miRs are modified to make them more resistant to degradation. pre-miRs have the same sense sequence to endogenous miRs and are precursor miRs that boost miR expression. miRs and pre-miRs can be delivered as oligonucleotides, for example, or after cloning into a vector such as a viral vector. Murine stem loop and mature sequences for miR-205 are CUCUUGUCCUUCAUUCCACCGGAGUCUGUCUUAUGCCAACCAGAUUUCAGUGGAGUGA AGCUCAGGAG (SEQ ID NO:1) and GAUUUCAGUGGAGUGAAGCUCA (SEQ ID NO:2), respectively. Murine stem loop and mature sequences for miR-411 are UGGUACUUGGAGAGAUAGUAGACCGUAUAGCGUACGCUUUAUCUGUGACGUAUGUAA CACGGUCCACUAACCCUCAGUAUCA (SEQ ID NO:3) and UAUGUAACACGGUCCACUAACC (SEQ ID NO:4), respectively. In these sequences, the mature sequence is the functional part of the miR. Human miR-205 and miR-411 sequences are known in the art.

When referring to an amino acid residue in a peptide, oligopeptide or protein, the terms “amino acid residue”, “amino acid” and “residue” are used interchangably and, as used herein, mean an amino acid or amino acid mimetic joined covalently to at least one other amino acid or amino acid mimetic through an amide bond or amide bond mimetic.

As used herein, “protein” and “polypeptide” are used synonymously to mean any peptide-linked chain of amino acids, regardless of length or post-translational modification, e.g., glycosylation or phosphorylation.

By the terms “nanoparticle” and “nanovehicle” is meant a microscopic particle whose size is measured in nanometers.

When referring to a nucleic acid molecule or polypeptide, the term “native” refers to a naturally-occurring (e.g., a wild-type (WT)) nucleic acid or polypeptide.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

The phrases “isolated” or biologically pure” refer to material (e.g., nucleic acids) which is substantially or essentially free from components which normally accompany it as found in its native state.

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

As used herein, the term “obesity” is defined as a medical condition in which excess body fat has accumulated and may have an adverse effect on health, leading to reduced life expectancy and/or increased health problems. Subjects with a body mass index (BMI), exceeding 30 kg/m² are considered as obese.

By the term “overweight” is meant an intermediate stage, defined as having more body fat than is optimally healthy but with a BMI less than 30 kg/m² that constitutes obesity.

By the phrases “therapeutically effective amount” and “effective dosage” is meant an amount sufficient to produce a therapeutically (e.g., clinically) desirable result; the exact nature of the result will vary depending on the nature of the disorder being treated. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

As used herein, the term “treatment” is defined as the application or administration of a therapeutic agent (e.g., a composition) described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, with human patients being preferred. In some cases, the methods described herein find use in experimental animals, in veterinary applications, and in the development of animal models for disease, including, but not limited to, rodents including mice, rats, and hamsters, as well as non-human primates.

Although compositions, kits and methods similar or equivalent to those described herein can be used in the practice or testing of the present invention, suitable compositions, kits and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a series of micrographs, heat maps and graphs showing diet-regulated adiopogenesis and related gene and micro-RNA expression, and metabolic profiles in NZ10 and SWR mice. (A) Increased density and heterogeneity of adipocytes in WD/NZ10 visceral adipose (VA) (B) Gene and micro-RNA heat map showing increased Mmp, Cathepsin D and K and angiogenic gene expression associated with decreased miR-205 in WD/NZ10 VA tissue (C-H) increased body weight (C) % fat (D) blood glucose (E) serum insulin (F) triglycerides (H) in WD/NZ10 group. (G) glucose tolerance test (I and J) gene expression of WD/NZ10 compared with WD/SWR.

FIG. 2 is a series of schematic illustrations, graphs, and micrographs showing miR-205-mediated regulation of adipogenesis in 3T3-L1 adipocytes. (A) Protocol for 3T3-L1 adipogenesis and determination of the role of miR-205 (B) Effect of miR-205 transfection on 3T3-L1 pre-adipocyte proliferation (C) endogenous levels of miR-205 during 3T3-L1 differentiation (D) Triglyceride levels (E) Oil Red-stained 3T3-L1 after differentiation in the presence of transfected pre-miR/anti-miR-205 as indicated. (F) Quantification of Oil Red stain from (E).

FIG. 3 is a heat map, a graph, a photograph and a schematic illustration showing regulation of gluconeogenesis by miR-411. (A) Micro-RNA heat map showing miR-411 reduced >3-fold in liver of NZ10 mice fed WD and increased ˜3-fold in SWR-fed MD. (B) Fold inductions of GN master transcription factors and enzyme genes in NZ 10-WD compared with NZ 10-MD livers. (C) Western blot of HEPG2 cells transfected with anti- or pre-MiR 411. (D) Schematic of gluconeogenesis master gene regulation.

### DETAILED DESCRIPTION

The compositions, methods and kits described herein may be used for treating and preventing obesity and overweight in a subject, and are based on the discovery that miR-205 expression is down-regulated in visceral adipose of obese/diabetic mice and may contribute to the hypertrophy and hyperplasia associated with visceral adiposity. In humans, central adiposity and associated adipogenic genes are strongly correlated with insulin resistance, Type 2 diabetes, dyslipidemia, and cardiovascular disease (CVD). To date, microRNA targets have only been identified in monogenic or diet-induced models of obesity and diabetes. These models involve severe obesity and hyperinsulinemia that is usually associated with hyperphagia and extreme leptin levels. There are no studies of the regulation of microRNAs in polygenic models of obesity-induced diabetes. The latter is much more relevant to the human condition because it entails complex interactions between genetic susceptibility and diet. The discovery described herein that NONcNZO10 mice fed a normal chow diet for 25 weeks become obese and diabetic creates a unique model for studies designed to understand the interaction between diet and genetics in the evolution of diabetosity. Presented herein is the first evidence that miR-205 expression is down-regulated in visceral adipose of obese/diabetic mice and may contribute to the hypertrophy and hyperplasia associated with visceral adiposity. In vitro studies using 3T3-L1 preadipocyte with miR-205 expression manipulated by pre and antago-miR transfection revealed significant induction of both preadipocyte cell proliferation and adipocyte lipid accumulation in the miR-205 knock down group. For the first time it has been shown that miR-205 expression increases as 3T3-L1 preadipocytes differentiate. It is hypothesized that a deficit of miR-205 in the visceral adipose of obese subjects simultaneously regulates the two key pathways (hyperplasia/hypertrophy) implicated in visceral adiposity.

The compositions, methods and kits described herein may also be used for treating and preventing hyperglycemia in a subject, and are based on the discovery that miR-411 is involved in the regulation of gluconeogenic genes by targeting master transcription factors. The expression of mir-411 was decreased in the liver of diabetic (NONcNZO10) mice after feeding a diabetic-diet for 15 weeks. The expression of miR-411 was normal when NONcNZO10 mice were fed a non-diabetic diet to maintain normal fasting glycemia, or in control mice that do not develop hyperglycemia under any diet. Delivery of pre-miR-411 to hepatocytes blocked the expression of gluconeogenic genes. By introducing higher levels of miR-411 into hepatocytes than would normally exist in a diabetic, repression of HNF4a is achieved, which in turn reduces gluconeogenesis. Agents that increase miR-411 expression, such as pre-miR-411, are thus novel candidate anti-diabetic agents that may be used to treat and resolve the symptoms and consequences of hyperglycemia.

The below described preferred embodiments illustrate adaptations of these compositions, kits and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Conventional methods of gene transfer and gene therapy may also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010.

### Compositions for Treating or Preventing Obesity, Overweight or Hyperglycemia in a Subject

Compositions for treating or preventing obesity, overweight or hyperglycemia in a subject are described herein. A composition for treating or preventing obesity or overweight in a subject includes at least one agent that increases expression of one or more miRs involved in adipogenesis, pre-adipocyte proliferation or differentiation, e.g., one or more of miR-200a, miR-200b, miR-200c, miR-143, and miR-205, in a therapeutically effective amount for treating or preventing obesity or overweight in a subject. Such a composition may include one agent that increases expression of one miR (e.g., miR-205). Alternatively, a composition may include two or more (e.g., 2, 3, 4, 5, 6) agents that increase expression of two or more miRs For example, a composition may include an agent that increases expression of miR-205 and an agent that increases expression of miR-200a. In another example, a composition may include an agent that increases expression of miR-205, an agent that increases expression of miR-200a, and an agent that increases expression of miR-200b. A composition for treating or preventing hyperglycemia in a subject includes an agent that increases expression of miR-411 in a therapeutically effective amount for treating or preventing hyperglycemia in a subject. The compositions also include a pharmaceutically acceptable carrier.

Any suitable agent for increasing expression of one or more of miR-200a, miR-200b, miR-200c, miR-143, and miR-205 (for treating or preventing obesity or overweight), and for increasing expression of miR-411 (for treating or preventing hyperglycemia) can be used. A suitable agent may be a nucleic acid, a chemical compound (e.g., a drug), a polypeptide, a peptide, etc. In the experiments described herein, a pre-miR-205 was shown to block proliferation of pre-adipocytes and adipogenesis, and thus in one embodiment, a composition for treating or preventing obesity or overweight in a subject includes a therapeutically effective amount of pre-miR-205 (e.g., pre-miR-205 delivered as an oligonucleotide or within a viral vector or nanoparticle) and a pharmaceutically acceptable carrier. In another embodiment, a composition for treating or preventing obesity or overweight in a subject includes a therapeutically effective amount of a vector (e.g., a viral vector) expressing miR-205 and a pharmaceutically acceptable carrier. In yet another embodiment, an agent that increases expression of miR-205 is a PPAR gamma antagonist. A composition for treating or preventing obesity or overweight in a subject may include two or more agents for increasing expression of a miR in a subject, e.g., a pre-miR to at least one miR as well as a vector expressing the miR Also in the experiments described herein, a pre-miR-411 was shown to block liver gluconeogenesis and thus in one embodiment, a composition for treating or preventing hyperglycemia in a subject includes a therapeutically effective amount of pre-miR-411 (e.g., pre-miR-411 delivered as an oligonucleotide or within a viral vector or nanoparticle) and a pharmaceutically acceptable carrier. A composition for treating or preventing hyperglycemia in a subject may additionally or alternatively include a vector (e.g., a viral vector) expressing miR-411.

In some embodiments, the compositions also include tissue-specific or cell-specific targeting molecules that recognize and bind cell surface ligands, including aptamers. For example, a composition for treating or preventing obesity or overweight in a subject may include an adipose cell surface-specific molecule. Examples of adipose cell-specific molecules include the adipose tissue-specific circular peptide (KGGRAKD) (SEQ ID NO:5) that can be combined with nanoparticles (including lipid nanoparticle complexes) or miRs and used to deliver miRs selectively to adipocytes (Hossen et. al., J Control Release. 2010, 147(2):261-8). Aptamers adipo-1 and adipo-8 described in Liu et al., (PLoS One. 2012;7(5):e37789) that can specifically recognize mature adipocytes may be included in a composition as described herein for cell- or tissue-specific targeting. Also, TDA1 (CGLHPAFQC) (SEQ ID NO:6), is a targeting ligand for visceral adipose tissue (described by Li et al, J Drug Target. 2011, 19(9):805-13) that may be included. In another example, a composition for treating or preventing hyperglycemia in a subject may include a liver-specific targeting molecule. For example, peptide derivatized-dendrimer, acetyl-CKNEKKNKIERNNKLKQPP-amide (SEQ ID NO:7) can be electrostatically complexed with specific miRs to selectively target heparin sulfate sites on the surface of hepatocytes. The peptide consists of amino acids 76-93 of the N-terminal region of the protozoan Plasmodium Berghei circumsporozoite protein (CSP) and mimics the liver-targeting strategy of this parasite. In one embodiment, miRs can be complexed with liver/hepatocyte-specific receptors such as the mannose-6-phosphate insulin-like growth factor II (M6P/IGFII)-receptor (van Beuge et al., Pharm Res. 2011 August; 28(8):2045-54).

Compositions for treating or preventing hyperglycemia, obesity or overweight in a subject can be administered in any suitable form. In one embodiment, the agent that increases expression of one or more of: miR-200a, miR-200b, miR-200c, miR-143, and miR-205 (for treating or preventing obesity or overweight), or miR-411 (for treating or preventing hyperglycemia) is a pre-miR, and is delivered to a subject in the form of naked chemically modified oligonucleotides. Chemically modified oligonucleotides as described herein generally contain complete phosphorothiolate backbones (for providing long-term stability and efficacy in vivo, see Boon et al., Circ Res. 2011 Oct. 28; 109(10):1115-9).

In some embodiments, such oligonucleotides can be formulated with nanoparticles. Examples of nanoparticles that may be particularly useful for delivering agents for increasing expression of miRNAs include (1) standard pre-miR complexed with lipid nanoparticles (DLin-KC2-DMA, distearoylphosphatidylcholine, cholesterol and mPEG2000-DMG) modified with peptide aptomers for specific and efficient delivery to liver or adipose tissue (Trajkovski et al., Nature 474: 649-53, 2011); (2) PEGylated LPH (liposome-polycation-hyaluronic acid) nanoparticle formulation again modified with peptide aptomers for specific and efficient delivery to liver or adipose tissue (Liu et al., Mol Pharm. 2011 Feb. 7; 8(1):250-9). In other embodiments, the pre-miR is cloned into a nucleic acid expression vector before being administered to a subject. Many vectors useful for introducing exogenous nucleic acids into target mammalian cells are available. The vectors may be episomal, e.g. plasmids, virus-derived vectors such cytomegalovirus, adenovirus, adeno-associated virus (AAV), lentivirus etc., or may be integrated into the target cell genome, through homologous recombination or random integration, e.g. retrovirus derived vectors such MMLV, HIV-1, ALV, etc. Various techniques using viral vectors for the introduction of nucleic acids (e.g., pre-miRs, miRs) into cells are provided for according to the compositions and methods described herein. Viruses are naturally evolved vehicles which efficiently deliver their genes into host cells and therefore are desirable vector systems for the delivery of therapeutic nucleic acids. Preferred viral vectors exhibit low toxicity to the host cell and produce/deliver therapeutic quantities of the nucleic acid of interest (in some embodiments, in a tissue-specific manner). Retrovirus-based vectors, Lentivirus vectors, adenovirus based vectors, and AAV-based vectors are examples of viral vectors that may be used.

### Methods of Treating or Preventing Obesity or Overweight and Treating or Preventing Hyperglycemia in a Subject

Described herein are methods of treating or preventing obesity or overweight in a subject. A typical method includes administering to the subject a composition including at least one agent that increases expression of one or more of: miR-200a, miR-200b, miR-200c, miR-143, and miR-205, in a therapeutically effective amount for treating or preventing obesity or overweight in the subject, and a pharmaceutically acceptable carrier. In the method, administration of the composition reduces adiposity levels and adipose production (reduces adipogenesis, decreases adipose mass) in the subject. Any suitable agent that increases expression of one or more of: miR-200a, miR-200b, miR-200c, miR-143, and miR-205 in the subject can be used, e.g., pre-miRs of one or more of: miR-200a, miR-200b, miR-200c, miR-143, and miR-205; a vector expressing one or more of these miRs. As described above, a composition may include two or more agents for increasing expression of two or more miRs in the subject. In a method of treatment, the subject (e.g., human) is overweight or obese. The subject may have a genetic predisposition to obesity. In some embodiments, the subject has type 2 diabetes or is pre-diabetic. In a method of preventing obesity or overweight in a subject, the subject may be at risk for obesity or overweight, e.g., may have a genetic predisposition to obesity. In these methods, the composition can be administered to the subject once, or a plurality of times as needed. They can be administered one or more times a day as needed.

Also described herein are methods of treating or preventing hyperglycemia in a subject. A typical method of treating or preventing hyperglycemia in a subject includes administering to the subject a composition including an agent that increases expression of miR-411 in a therapeutically effective amount for treating (e.g., eliminating, reducing) or preventing hyperglycemia. In the method, administration of the composition reduces tissue glucose production and increases glucose removal in the subject. Generally, administration of the composition also reduces hepatic HNF4a expression in the subject. Any suitable agent that increases expression of miR-411 in the subject can be used, e.g., a pre-miR-411, a vector expressing miR-411. In a method of treatment, the subject (e.g., human) is hyperglycemic. The subject may also have a genetic predisposition to obesity, type 2 diabetes or be pre-diabetic, obese, or overweight. The subject may be suffering from or at risk of one or more of: acute hyperglycemia, chronic hyperglycemia, and stress hyperglycemia. Typically, the subject has hyperglycemia that is associated with obesity and/or type 2 diabetes. In a method of preventing hyperglycemia in a subject, the subject may be at risk for hyperglycemia, e.g., may have one or more risk factors for hyperglycemia such as dyslipidemia, pre-diabetes, elevated blood triglycerides, metabolic syndrome, and obesity. In these methods, the composition can be administered to the subject once, or a plurality of times as needed. They can be administered one or more times a day as needed.

In these methods, the compositions can be administered to a subject by any suitable route, e.g., systemically by intravenous injection, directly to a target site, etc. For example, in a method of treating or preventing hyperglycemia, a composition as described herein may be delivered systemically or more directly to a subject's liver by injection through the portal vein. In a method of treating or preventing obesity or overweight, a composition may be administered directly to a subject's visceral adipose tissue. The therapeutic methods described herein in general include administration of a therapeutically effective amount of the compositions described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider. The methods and compositions herein may be also used in the treatment of any other disorders in which downregulation of microRNAs may be implicated.

In one embodiment, a method of treating hyperglycemia, obesity or overweight in a subject includes monitoring treatment progress. Monitoring treatment progress in a subject generally includes determining a measurement of hyperglycemia, obesity or overweight or other diagnostic measurement in a subject having hyperglycemia, obesity or overweight, prior to administration of a therapeutic amount of a composition as described herein sufficient to treat hyperglycemia, obesity or overweight in the subject. At one or more time points subsequent to the subject having been administered a therapeutic amount of a composition as described herein, a second measurement of hyperglycemia, obesity or overweight is determined and compared to the first measurement of hyperglycemia, obesity or overweight. The first and subsequent measurements are compared to monitor the course of the disease or disorder (hyperglycemia, obesity or overweight) and the efficacy of the therapy.

### Kits

Described herein are kits for treating or preventing obesity or overweight in a mammalian subject. A typical kit for treating or preventing obesity or overweight in a mammalian subject includes a therapeutically effective amount of a composition including at least one agent (e.g., a pre-miR, a vector expressing a miR) that increases expression of one or more of: miR-200a, miR-200b, miR-200c, miR-143, and miR-205 in a therapeutically effective amount for treating or preventing obesity or overweight in the subject, and a pharmaceutically acceptable carrier, with instructions for administering the composition to the subject. Also described herein are kits for treating hyperglycemia, particularly hyperglycemia associated with obesity and/or type 2 diabetes. Such a kit typically includes an agent that increases expression of miR-411 (e.g., a pre-mir-411, a vector expressing miR-411) in a subject in a therapeutically effective amount for treating or preventing hyperglycemia, with instructions for administering the composition to the subject. In a kit, the instructions generally include one or more of: a description of the composition; dosage schedule and administration for treatment of obesity and/or overweight or for treatment of hyperglycemia; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### Administration of Compositions

The compositions described herein may be administered to mammals (e.g., rodents, humans, nonhuman primates, canines, felines, ovines, bovines) in any suitable formulation. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to mammals by any conventional technique. When treating a subject having obesity, overweight, or hyperglycemia, the composition may be administered to the subject intravenously, directly into a target tissue, parenterally, orally, etc. The compositions may be administered directly to a target site by, for example, surgical delivery to an internal or external target site, or by catheter to a site accessible by a blood vessel. For example, a composition for treating or preventing hyperglycemia as described herein may be delivered directly to the liver. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Effective Doses

The compositions described herein are preferably administered to a mammal (e.g., human) in an effective amount, that is, an amount capable of producing a desirable result in a treated mammal (e.g., treating or preventing obesity, overweight, or hyperglycemia). Such a therapeutically effective amount can be determined according to standard methods. Toxicity and therapeutic efficacy of the compositions utilized in methods of the invention can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently. In embodiments in which the agent that increases expression of a miR is a pre-miR and is an oligonucleotide, the oligonucleotides can be delivered with or without nanoparticles and targeting molecules and/or after incorporation into viral vectors (e.g., recombinant AAV (rAAV)). For naked oligonucleotides, the range for systemic delivery is approximately 0.5 to about 5 mg/kg. A delivery dose of nanoparticles and rAAV is determined based on preclinical efficacy and safety.

### EXAMPLES

The present invention is further illustrated by the following specific examples. The examples are provided for illustration only and should not be construed as limiting the scope of the invention in any way.

### Example 1

### Anti-Obesity Therapy Using Pre-Micro-RNA 205 to Block Adipogenesis and Pre-Adipocyte Proliferation

The role of miR-205 in the regulation of adipogenesis and adipose hypertrophy was identified. miR-205 expression is markedly repressed in VA of obese/diabetic NONcNZO10 mice after feeding a western style/diabetic-diet for 15 weeks. The expression of miR-205 was normal when NONcNZO10 mice were fed non-diabetic diet to maintain normal VA levels, or in control mice that do not develop excess VA under any diet. Delivery of pre-miR-205 to 3T3L1 pre-adipocytes blocked both proliferation and adipogenesis whereas delivery of antagomir-205 promoted adipocyte proliferation, differentiation and lipid production. miR-205 may be targeted as a new therapy for the treatment/prevention of obesity especially in subjects with a genetic predisposition.

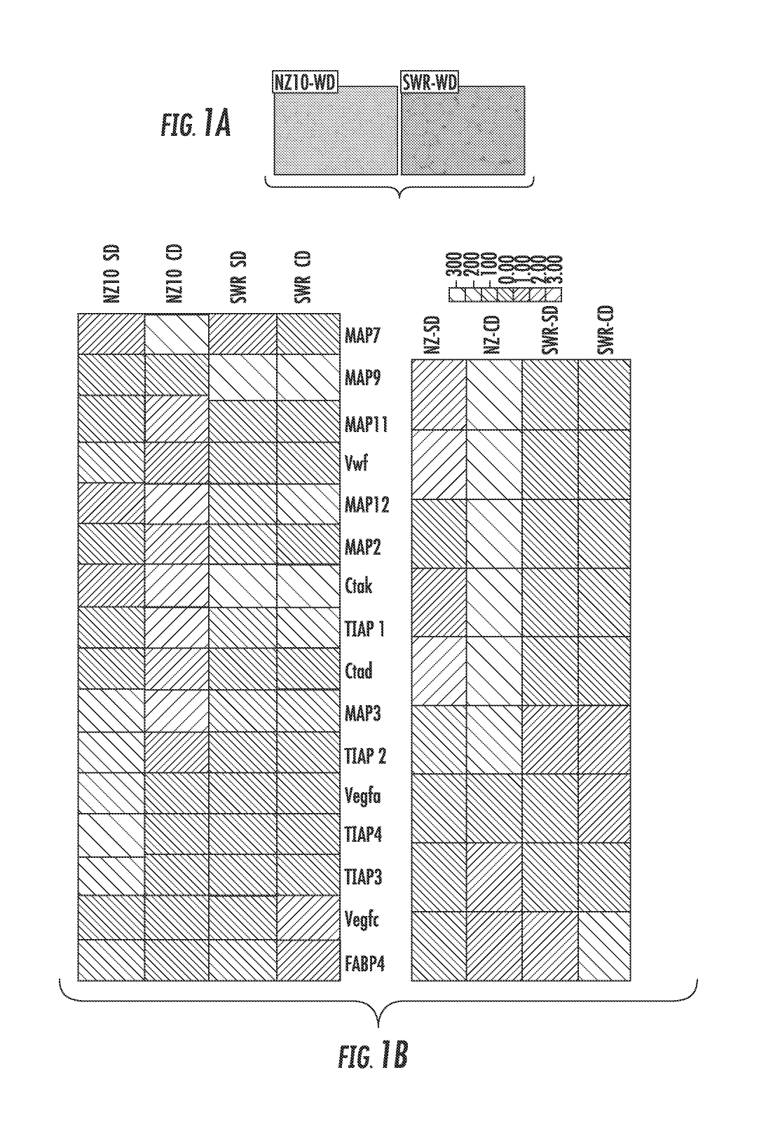

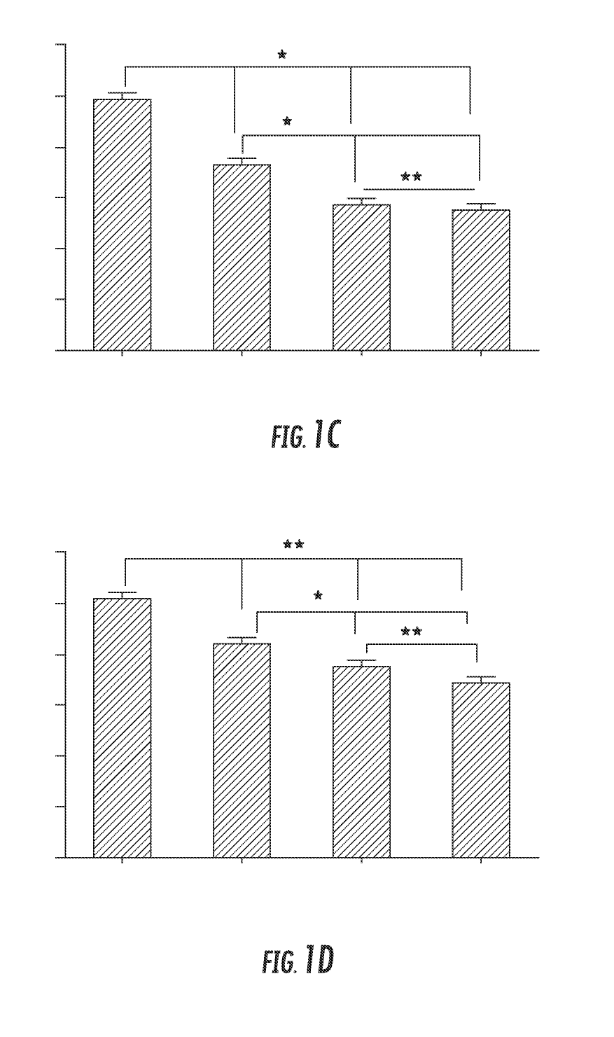

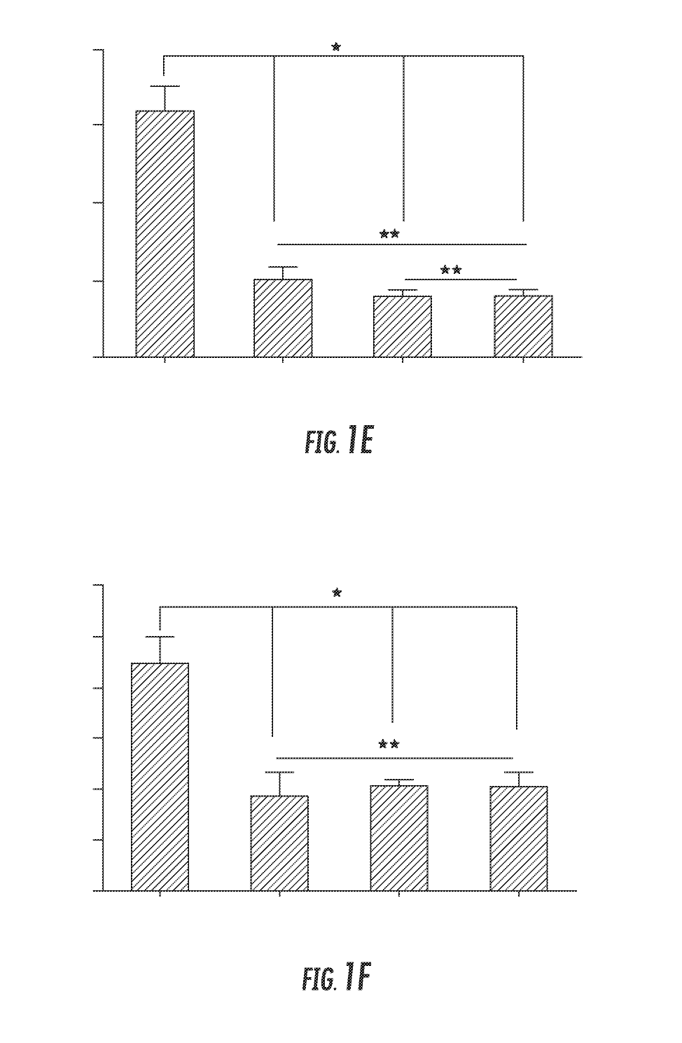

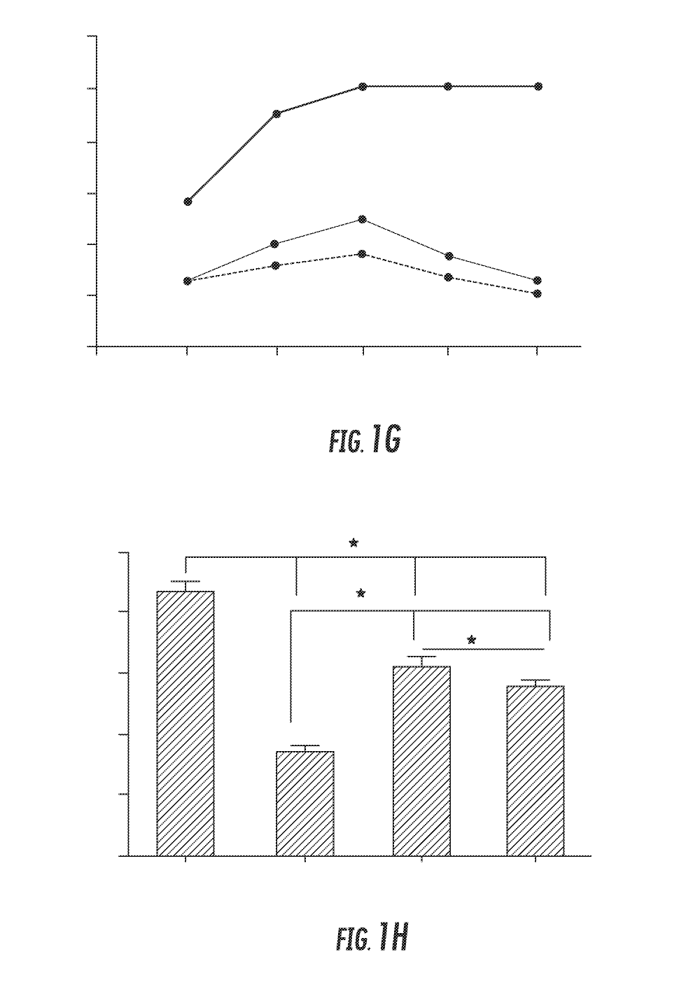

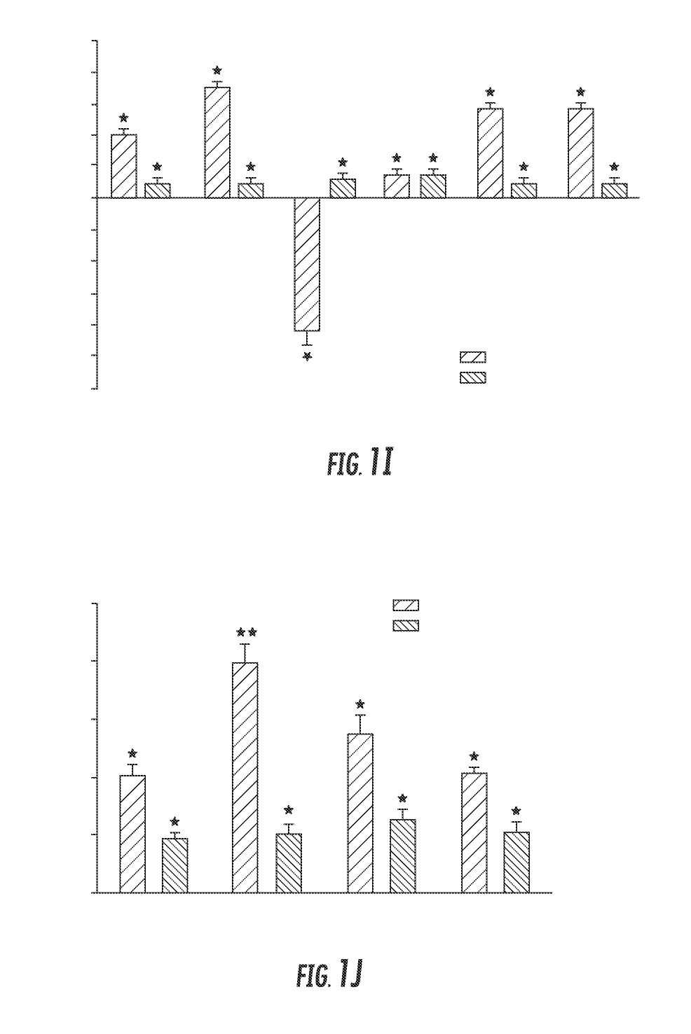

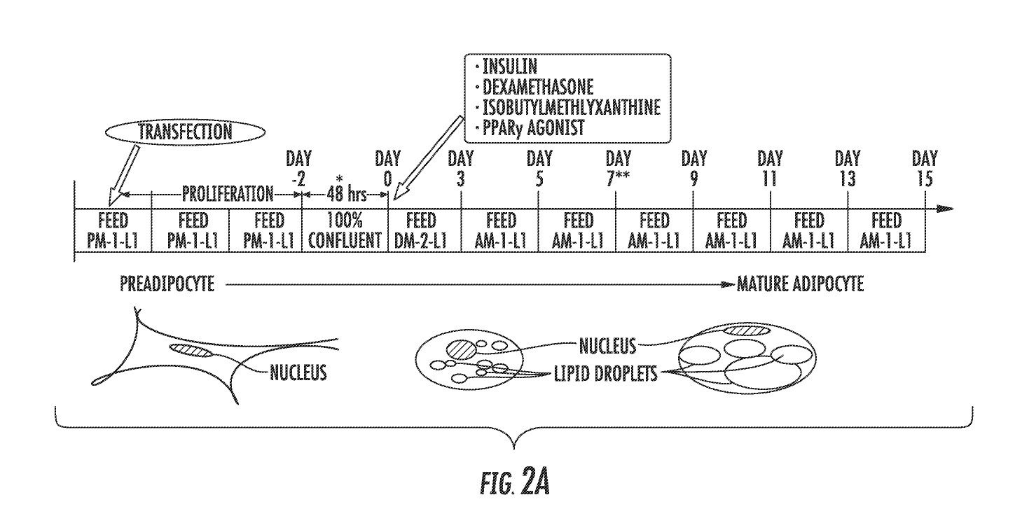

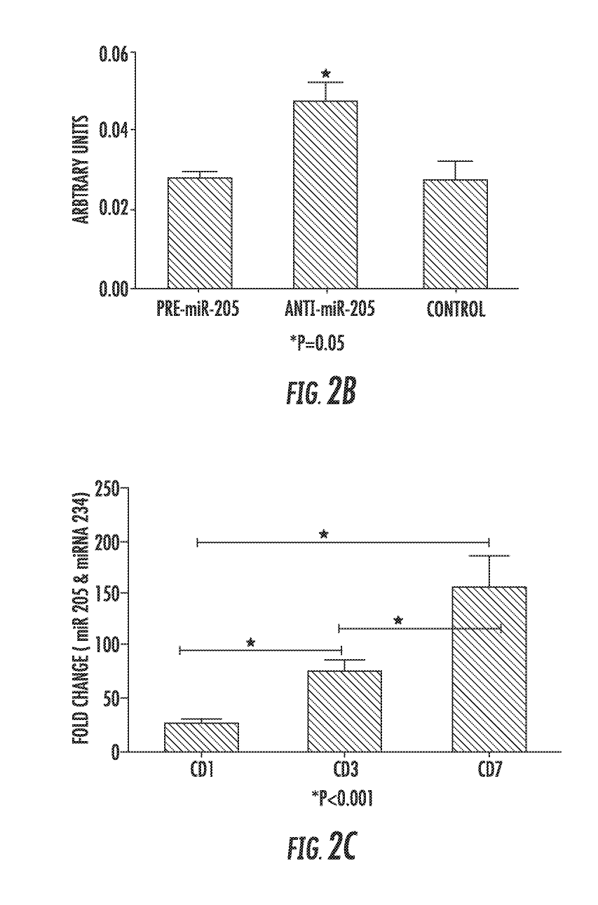

Regulation of adipogenesis by miR-205: Male NZ10 and SWR control mice obtained from Jackson Labs (60 per group) were fed Western (WD; high fat/carb) or Mediterranean (MD; high protein/unsaturated fat) diets for 15 weeks. SWR mice retained normal glycemic control and blood chemistry over 15 weeks on both diets; WD-fed NZ10 mice had increased plasma triglycerides (3-fold, p<0.001; maximum 340 mg/dL) insulin (1.8-fold. P<0.01) and severely impaired glucose tolerance relative to the MD or control groups (p<0.01). The mean resting and fasting glucose of NZ10 mice fed WD were 450 mg/dL and 350 mg/dL respectively. Euglycemic/hyperinsulinemic clamps confirmed whole body insulin resistance of NZ10 mice. NZ10 mice accumulated significantly more VA than SWR controls. Body weights of WD-fed NZ10 mice were 18% greater and fat depots 22±3.1% larger than MD mice (p<0.01) (see FIG. 1). For micro-RNA and gene expression micro-arrays, RNA pools from 5 mice per group were hybridized with a mouse panel of 640 transcripts or complete Affymetrix mouse gene expression arrays. Several miRs were identified that were responsive to diet, genetic background or both. As illustrated in FIG. 1B, miR-205 expression was strongly genetics- and diet-dependent; miR-205 expression was decreased ˜10-fold in NZ10 mice fed WD compared with SWR fed MD (confirmed by rt-PCR). Adipogenesis-related genes including metalloproteases (Mmps), cathepsins (Cts D and K) and angiogenic genes (VEGF, Vwf) were upregulated in parallel. To confirm the role of mir-205 in regulating adipogenesis, premiR-205 or antagomir-205 were transfected into mouse 3T3L1 pre-adipocytes and proliferation, differentiation and lipid production were measured. The experimental protocol is shown in FIG. 2A. As shown in FIG. 2B, miR-205 inhibition significantly increased cell proliferation of 3T3-L1 preadipocytes. Triglycerides were measured in the cell supernatant 48 h after transfection. Anti-miR-205 decreased triglyceride levels in the media consistent with increased lipogenesis. Oil Red O staining was performed on day 4 after induction of adipogenesis. miR-205 inhibition significantly increased lipid staining by oil red O suggesting increased adipogenesis (FIG. 2E). The lipid bound oil red O stain was extracted using dye extraction solution and quantified spectrophotometrically. FIG. 2F shows significantly increased levels of oil red O stain in the anti-miR-205 group. The miR-205 levels were quantitated at different time points during adipocyte differentiation and it was found that miR-205 expression increased in differentiated adipocytes relative to preadipocytes (FIG. 2C). Together, these results indicate (1) miR-205 negatively regulates pre-adipocyte proliferation; (2) miR-205 negatively regulates adipogenesis of 3T3-L1 adipocytes; (3) miR-205 levels increase coincident with adipogenesis; and (4) low expression of miR-205 in VA tissue in vivo correlates with excessive VA accumulation and an obese phenotype. These results suggest that reduced expression of miR-205 may be a cause of obesity and by extension, correction of miR-205 levels by systemic delivery of pre-miR-205 may provide a new approach to treat obesity. Targeted delivery of pre-miR-205 to VA may be an optimal technique to prevent obesity in subjects with a predisposition to overeating and/or obesity.

### Example 2

### Anti-Diabetes Therapy Using Pre-Micro-RNA 411 to Block Liver Gluconeogenesis and Normalize Glycemia

The role of miR411 in regulation of gluconeogenic genes by targeting master transcription factors was identified. The expression of mir-411 was decreased in the liver of diabetic (NONcNZO10) mice after feeding a diabetic-diet for 15 weeks. The expression of miR-411 was normal when NONcNZO10 mice were fed a non-diabetic diet to maintain normal fasting glycemia, or in control mice that do not develop hyperglycemia under any diet. Delivery of pre-miR-411 to hepatocytes blocked the expression of gluconeogenic genes. By introducing higher levels of miR-411 into hepatocytes than would normally exist in a diabetic, repression of HNF4a is achieved, which in turn reduces gluconeogenesis.

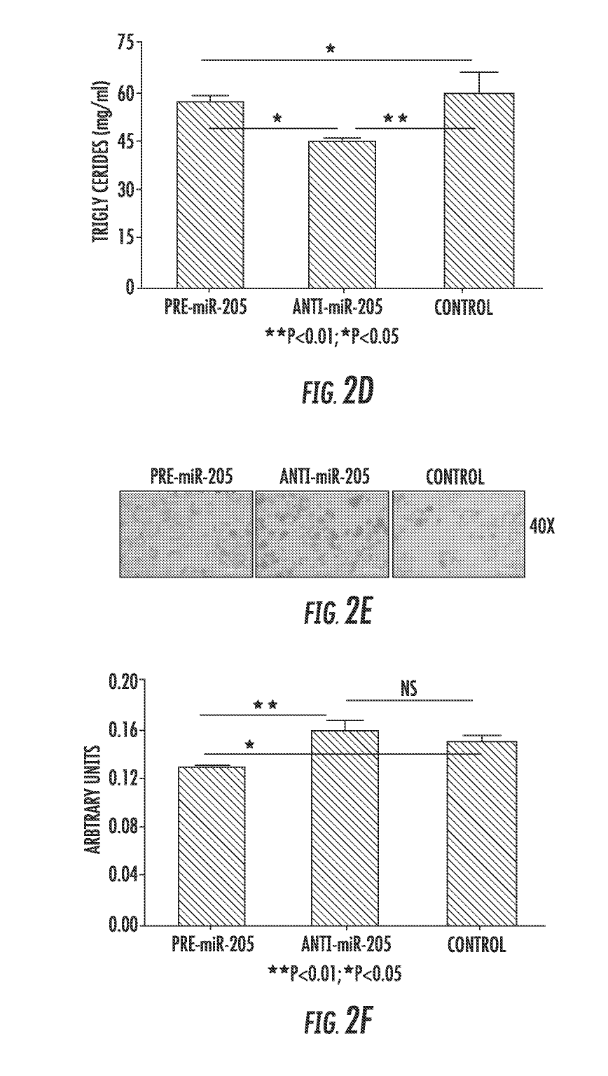

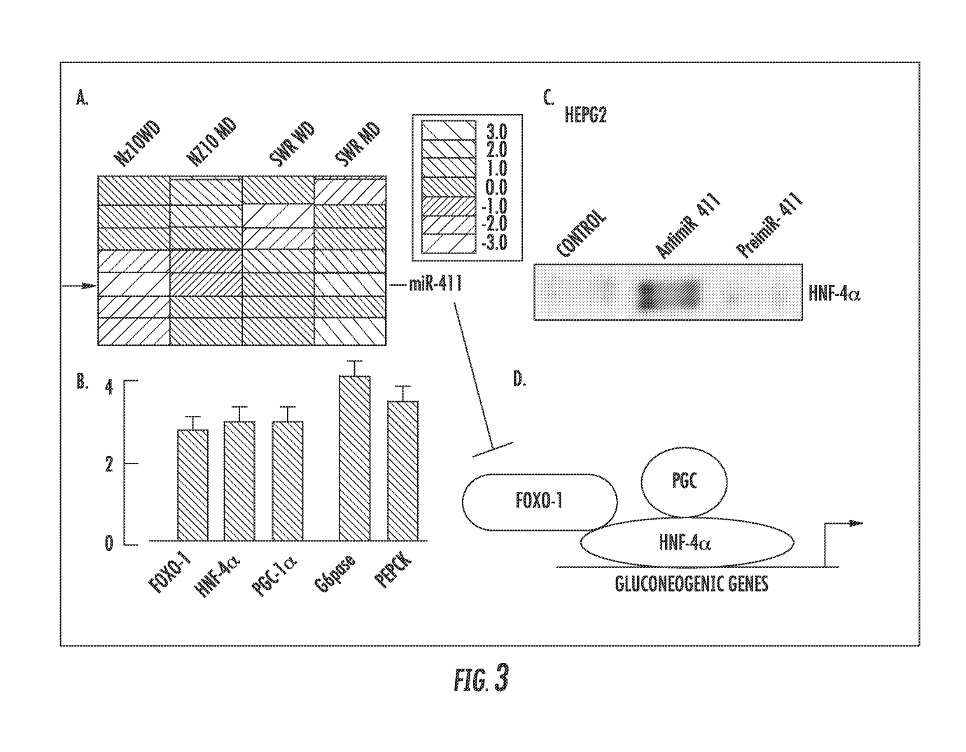

Regulation of gluconeogenesis by miR-411: Male NZ10 and SWR control mice obtained from Jackson Labs (60 per group) were fed WD or MD diets for 15 weeks. SWR mice retained normal glycemic control and blood chemistry over 15 weeks on both diets; WD-fed NZ10 mice had increased plasma triglycerides (3-fold, p<0.001; maximum 340 mg/dL) insulin (1.8-fold. P<0.01) and severely impaired glucose tolerance relative to the MD or control groups (p<0.01). The mean resting and fasting glucose of NZ10 mice fed WD were 450 mg/dL and 350 mg/dL, respectively. Euglycemic/hyperinsulinemic clamps confirmed whole body insulin resistance of NZ10 mice. NZ10 mice accumulated significantly more visceral adipose than SWR controls. Body weights of WD-fed NZ10 mice were 18% greater and fat depots 22±3.1% larger than MD mice (p<0.01). The serum levels of resistin, MCP-1 and insulin were increased in NZ10 mice fed the WD. For micro-RNA and gene expression micro-arrays, RNA pools from 5 mice per group were hybridized with a mouse panel of 640 transcripts or complete Affymetrix mouse gene expression arrays. Several miRs that were responsive to diet, genetic background or both were identified. As illustrated in FIG. 3, miR-411 expression was strongly genetics and diet-dependent; miR-411 expression was decreased ˜10-fold in NZ10 mice fed WD compared with SWR fed MD (confirmed by rt-PCR). FOXO-1 was identified as a target of miR-411 and increased FOXO-1 expression in WD-fed NZ10 mouse livers was confirmed by RT-PCR and western blot. RT-PCR also confirmed 2-4-fold increased expression of the gluconeogenic master transcription factors HNF-4a and PGC-1a as well as the gluconeogenic enzyme genes G6Pase (rate limiting) and PEPCK (FIG. 3B). To confirm the role of mir-411 in regulating gluconeogenesis, premiR-411 or antagomir-411 were transfected into human HEPG2 hepatocytes and HNF4a expression was measured. As shown in FIG. 3C, expression of HNF4a was increased by the antagomir and reduced by pre-miR-411. These results identify miR-411 as a regulator of gluconeogenesis that is dysregulated in a genetics and diet-dependent manner in mice with polygenic susceptibility for obesity and type 2 diabetes. As such, pre-miR-411 is a novel candidate anti-diabetic agent that may be used to treat and resolve the symptoms and consequences of hyperglycemia.

### Other Embodiments

Any improvement may be made in part or all of the compositions, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of treating or preventing obesity or overweight in a subject, the method comprising administering to the subject a composition comprising at least one agent that increases expression of at least one microRNA (miR) selected from the group consisting of: miR-200a, miR-200b, miR-200c, miR-143, and miR-205 in a therapeutically effective amount for treating or preventing obesity or overweight in the subject, and a pharmaceutically acceptable carrier, wherein administration of the composition reduces adiposity levels and adipose production in the subject.

2. The method of claim 1, wherein the at least one agent increases expression of miR-205 and is pre-miR-205.

3. The method of claim 2, wherein the pre-miR-205 is an oligonucleotide.

4. The method of claim 2, wherein the pre-miR-205 is comprised within a viral vector or a nanoparticle.

5. The method of claim 1, wherein the at least one agent that increases expression of at least one miR is a vector expressing the at least one miR.

6. The method of claim 5, wherein the vector is a viral vector and expresses miR-205.

7. The method of claim 1, wherein the composition further comprises a tissue-specific or cell-specific targeting molecule.

8. The method of claim 1, wherein the subject is human and is overweight or obese.

9. The method of claim 1, wherein the subject is a human and has a genetic predisposition to obesity.

10. The method of claim 1, wherein the subject is a human and has type II diabetes.

11. A composition comprising at least one agent that increases expression of at least one miR selected from the group consisting of: miR-200a, miR-200b, miR-200c, miR-143, and miR-205 in a subject in a therapeutically effective amount for treating or preventing obesity or overweight in a subject, and a pharmaceutically acceptable carrier, wherein the at least one agent comprises a viral vector expressing a pre-miR to the at least one miR or a viral vector expressing the at least one miR.

12. The composition of claim 11, wherein the at least one agent increases expression of miR-205 and is a viral vector expressing a pre-miR to miR-205.

13. The composition of claim 12, wherein the viral vector is comprised within a recombinant virion.

14. The composition of claim 11, wherein the at least one agent increases expression of miR-205, and the composition comprises a second agent that increases expression of miR-205, wherein the second agent is a PPAR gamma antagonist.

15. A method of treating or preventing hyperglycemia in a subject comprising administering to the subject a composition comprising an agent that increases expression of miR-411 in the subject in a therapeutically effective amount for treating or preventing hyperglycemia, wherein administration of the composition reduces tissue glucose production and increases glucose removal in the subject.

16. The method of claim 15, wherein administration of the composition reduces hepatic HNF4a expression in the subject.

17. The method of claim 15, wherein the composition is administered to the subject's liver.

18. The method of claim 15, wherein the subject is obese or overweight.

19. The method of claim 15, wherein the subject has diabetes or is pre-diabetic.

20. The method of claim 15, wherein the subject has at least one of: acute hyperglycemia, chronic hyperglycemia, and stress hyperglycemia.

21. The method of claim 15, wherein the at least one agent that increases expression of miR-411 is a vector expressing miR-411.

22. The method of claim 21, wherein the vector is a viral vector.

23. The method of claim 15, wherein the agent that increases expression of miR-411 is pre-miR-411, and wherein the pre-miR-411 is an oligonucleotide.

24. The method of claim 23, wherein the pre-miR is comprised within a viral vector or a nanoparticle.

25. The method of claim 15, wherein the composition further comprises a tissue-specific or cell-specific targeting molecule.

26. A composition comprising at least one agent that increases expression of miR-411 in a subject in a therapeutically effective amount for treating or preventing hyperglycemia in a subject, and a pharmaceutically acceptable carrier, wherein the at least one agent comprises a viral vector expressing pre-miR-411 or a viral vector expressing miR-411.


SEQUENCE LISTING (7 sequences)

SEQ ID NO: 1 - RNA, 68 nt, Mus musculus
cucuuguccuucauuccaccggagucugucuuaugccaaccagauuucaguggagugaagcucaggag

SEQ ID NO: 2 - RNA, 22 nt, Mus musculus
gauuucaguggagugaagcuca

SEQ ID NO: 3 - RNA, 82 nt, Mus musculus
ugguacuuggagagauaguagaccguauagcguacgcuuuaucugugacguauguaacacgguccacuaacccucaguauca

SEQ ID NO: 4 - RNA, 22 nt, Mus musculus
uauguaacacgguccacuaacc

SEQ ID NO: 5 - protein, 7 aa, Homo sapiens
KGGRAKD

SEQ ID NO: 6 - protein, 9 aa, Homo sapiens
CGLHPAFQC

SEQ ID NO: 7 - protein, 19 aa, ARTIFICIAL SEQUENCE
  SYNTHETIC CONSTRUCT
CKNEKKNKIERNNKLKQPP